(19) United States
(12) Patent Application Publication
(10) Pub. No.: US 2023/0390443 A1
(43) Pub. Date: Dec. 7, 2023

(54) APPARATUS FOR PROTECTING AND EXTENDING THE LIFE OF A FACEMASK

(71) Applicant: Robert Dallas Farrow, Austin, TX (US)

(72) Inventor: Robert Dallas Farrow, Austin, TX (US)

(21) Appl. No.: 18/324,030

(22) Filed: May 25, 2023

Related U.S. Application Data
(60) Provisional application No. 63/348,819, filed on Jun. 3, 2022.

(51) Int. Cl. A61L 2/22; A61L 2/10

(52) U.S. Cl. CPC A61L 2/22; A61L 2/10; A61L 2202/15; A61L 2202/122; A61L 2202/26

(57) ABSTRACT

An apparatus and a method of use therefore for treating used and new facemasks to extend their useful life. The method includes the steps of spraying antimicrobial solution onto a mask mounted within a compartment of the apparatus. After sufficient contact time, the mask can be dried off. Ultraviolet irradiation can also be used to synergistically sterilize the mask.

### CROSS-REFERENCE TO RELATED APPLICATIONS

This application claims priority from a U.S. Provisional Patent Application Ser. No. 63/348,819, filed on Jun. 3, 2022, the disclosure of which is incorporated herein by reference in their entirety.

### FIELD OF INVENTION

The present invention relates to an apparatus for extending the life of a facemask, and more particularly, the present invention relates to an apparatus that treats a facemask for extending its useful.

### BACKGROUND

Facemasks are being widely used nowadays for limiting the spread of contagious microbes. Covid-19 global pandemic increased the awareness and the use of masks in day-to-day settings for all people and all professional industries. Face masks and other personal protective equipment have been made essential for healthcare workers. The emergence and re-emergence of viral infectious diseases keep the demand for facemasks high. Covid-19 pandemic has drove awareness for the different types of masks, mask materials, mask filtration ratings, and mask filtration limitations.

The use of facemasks has certain drawbacks as well. Face masks are costly and often become a financial burden for any organization. Besides the cost, the use of masks generates a lot of waste which is hard to decompose. The Covid-19 pandemic which happened in 2019 is estimated to generate up to 7,200 tons of medical waste every day, much of which is disposable masks. In case of pandemics, the demand surges, and the supply remains limited resulting in shortages and unavailability. Thus, the mask production and logistics has become a global challenge.

Extending the useful life of the mask may help overcome many of the aforesaid drawbacks and limitations to a great extent. A need is therefore appreciated for a system that can increase the usable life of a mask. A need is also there for a system that can enhance the effectiveness of the mask.

The term mask, facemask, and face coverings are interchangeably used hereinafter. The facemask refers to a medical face mask (also known as a surgical or procedure mask), which is a medical device covering the mouth, nose, and chin, ensuring a barrier that limits the transition of an infective agent between the hospital staff and the patient. The face covering refers to a non-medical device that has the same design as the medical mask but made from materials that are not deemed to be highly effective by medical standards. This would include face coverings with a mask design made from cotton, silk, and other organic and inorganic textiles. The face masks or face coverings have different recommended uses for protection by governing authorities on a global level. For example, the professional industries have different needs of protection from chemical odors, gases, viruses and general pathogens.

### SUMMARY OF THE INVENTION

The following presents a simplified summary of one or more embodiments of the present invention to provide a basic understanding of such embodiments. This summary is not an extensive overview of all contemplated embodiments and is intended to neither identify critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to an apparatus for treating a mask for increasing its useful life.

It is another object of the present invention that the apparatus is compact for use in limited spaces.

It is still another object of the present invention that the apparatus is economical to manufacture and use.

It is yet another object of the present invention that the apparatus is easy to use.

It is a further object of the present invention that masks are safe and effective for further use.

It is still a further object of the present invention that the masks are sterilized/treated within a short duration.

It is yet a further object of the present invention to provide a portable version of the apparatus.

It is still a further object of the present invention that a used mask can be cleaned by the apparatus.

It is yet a further object of the present invention that the apparatus allows making new masks more effective by different coatings with for example, sanitizers, antimicrobials, disinfectants, and UV light solutions.

In one aspect, disclosed are an apparatus and method for extending the useful life of a used mask. The apparatus comprising a compartment having inner walls and a door, the inner walls comprise a bottom wall, a top wall, and side walls; hooks and/or clips coupled to the inner walls of the compartment; a plurality of nozzles configured to spray antimicrobial solution, the plurality of nozzles disposed within the compartment; one or more fans to blow air disposed within the compartment; and one or more heating elements to heat the air within the compartment. The method comprises mounting a mask to the hooks and/or clips; spraying the antimicrobial solution over the mask through one or more of the plurality of nozzles; and upon spraying and after a predefined duration, drying the mask using hot air from the one or more fans.

In one aspect, the apparatus further comprises one or more ultraviolet lights disposed within the compartment, the method further comprises irradiating the mask with UV light from the one or more ultraviolet lights.

In one aspect, the compartment has vent holes for the air to egress from the compartment. The top portion of the compartment is slanted in design to prevent the build-up of moisture. The apparatus further comprises a reservoir containing the antimicrobial solution, the reservoir connected to the plurality of nozzles.

### BRIEF DESCRIPTION OF THE DRAWINGS

The accompanying figures, which are incorporated herein, form part of the specification and illustrate embodiments of the present invention. Together with the description, the figures further explain the principles of the present invention and enable a person skilled in the relevant arts to make and use the invention.

FIG. 1 is a schematic view of a compartment of the apparatus, according to an exemplary embodiment of the present invention.

FIG. 2 shows the apparatus according to an exemplary embodiment of the present invention.

### DETAILED DESCRIPTION

Subject matter will now be described more fully hereinafter with reference to the accompanying drawings, which form a part hereof, and which show, by way of illustration, specific exemplary embodiments. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, the reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is to describe particular embodiments only and is not intended to be limiting of embodiments of the invention. As used herein, the singular forms “a”, “an”, and “the” are intended to include the plural forms as well, unless the context indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely to illustrate the general principles of the invention since the scope of the invention will be best defined by the allowed claims of any resulting patent.

An apparatus is disclosed for treating used masks for extending their useful life. The used masks treated with the disclosed apparatus can be used beyond their prescribed use life, safely and efficiently. The masks can easily be treated in the disclosed apparatus, wherein the mask can be cleaned and sterilized in a short duration. The disclosed apparatus provides synergistic sterilizing conditions that efficiently conserve the used mask in a short duration. The disclosed apparatus can be used for both disposable and reusable masks. The disclosed apparatus can also provide for enhancing effectiveness of new masks by suitable coating process.

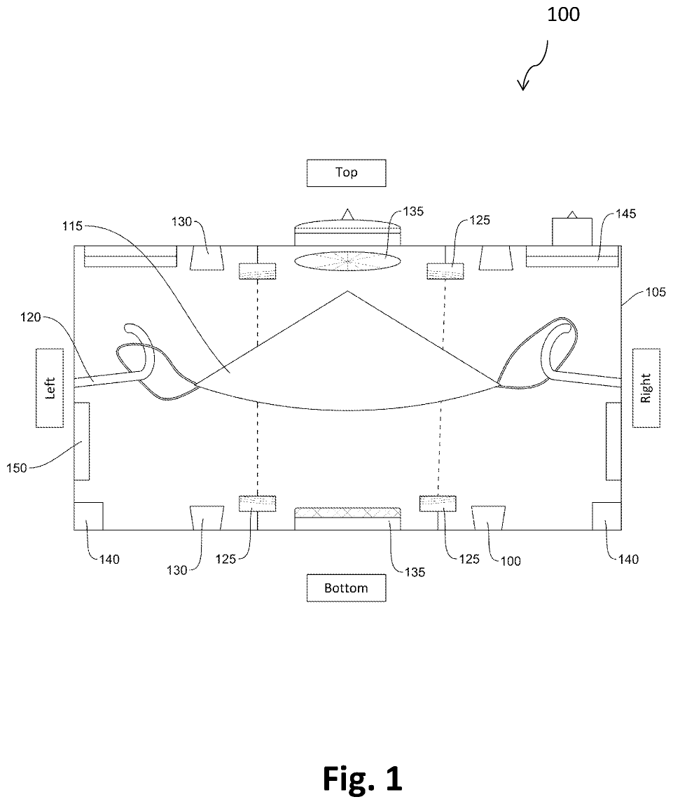

Referring to FIG. 1 shows an embodiment of the disclosed apparatus 100. The disclosed apparatus has an enclosed compartment 105 that has inner walls and a door, the door provides access to the compartment. The compartment can be made from a material that can be strong and rust-free so that it can withstand the environmental conditions inside the compartment, as described below. For example, the compartment can be made from aluminum or stainless steel. The compartment can be sealed by the door. The shape and volume of the compartment can vary without departing from the scope of the present invention. For example, the compartment can be made spherical, or box shaped. The size of the compartment may depend upon the number of masks that need to be simultaneously treated. The door can be coupled to the compartment through hinge joints. Alternatively, the door can be slidably coupled to the compartment. In either case, a handle can be provided to pull or slide the door for opening and closing the compartment. A suitable lock can also be provided to secure the door in a locked state while operating the apparatus. Different controls of the apparatus can be provided on the outside of the compartment or upon the door. Suitable safety sensors can also be incorporated to indicate that the door has been properly closed.

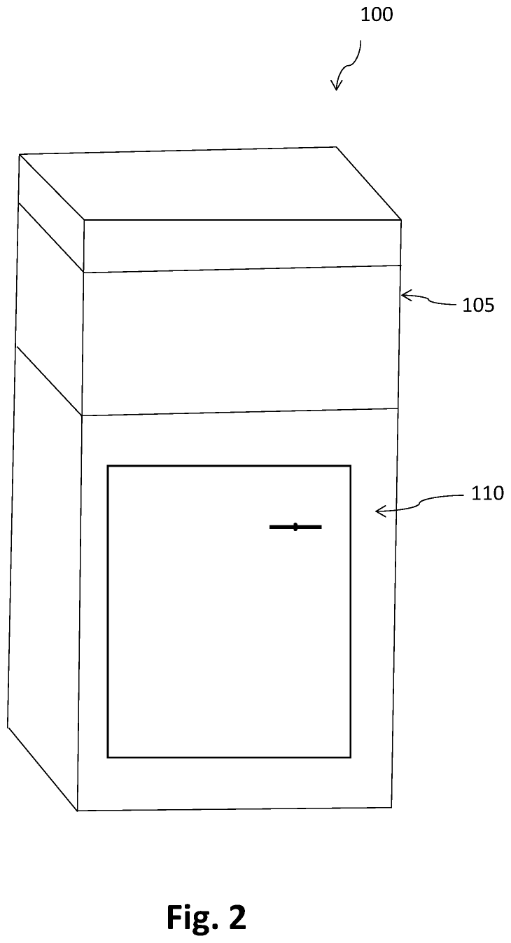

FIG. 2 shows an exemplary embodiment of the disclosed apparatus 100 that is floor mounted. Compartment 105 at the top is of a cuboidal shape and another storage compartment 110 is provided below the compartment. The storage compartment can be used to store different articles including masks. The bottom compartment can also be used as a recycling compartment. The bottom compartment can however be optional.

The bottom compartment may also store reservoirs, such that the reservoir for antimicrobial solution for the disclosed apparatus. Such an antimicrobial solution can be pump-fed or gravity fed. The height of the main compartment above the ground can be such that the inside of the compartment is reachable to a person of an average height for placing and removing masks from the main compartment.

Several hooks or clips can be provided on the inner walls of the compartment. From the hooks, the mask can be hung for treatment in the apparatus. The mask can be hung from the strap or loop of the mask that is used to secure the mask around the head or ears. Preferably, as shown in FIG. 1, mask 115 can extend between two walls of the compartment i.e., the mask can be engaged to hooks 120 on adjacent or opposite walls of the compartment. This way, the maximum surface area of the mask can be exposed to the treatment agents in the disclosed apparatus. For example, the antimicrobial solution can be uniformly sprayed over the mask that is hung from the inner walls of the compartment. Clips 125 can also be provided for securing the mask.

The antimicrobial solution can be used to disinfect the mask, wherein the antimicrobial solution can be sprayed using suitable nozzles 130 disposed within the compartment of the disclosed apparatus. One or more nozzles connected to an antimicrobial solution reservoir can spray the antimicrobial solution over the mask secured within the apparatus. The nozzles can be positioned such that the antimicrobial solution can be sprayed directly on the mask without any wastage. The pattern of the spray can be varied by changing the configuration of the nozzles. Preferably, the antimicrobial solution can be sprayed in the form of a mist to prevent wet spots in the mask.

The masks sprayed with the antimicrobial solution can be left for a desired contact period for the antimicrobial to have its action. Thereafter, the mask can be quickly dried off using hot air. One or more fans 135 can be suitably placed within the compartment for blowing air over the mask for quickly drying the same. The spraying and drying cycles can be repeated one or more times to affect the sterilization of the mask. Moreover, the time between spraying and the drying and the drying time can also be varied in consecutive cycles, so that the overall process can be completed quickly and efficiently. Suitable vent holes can also be incorporated for the air inside the compartment to be blown out. The air containing moisture from the mask can exhaust through these vent holes. Also, suitable drainage along the floor of the compartment can also be incorporated so that any condensed liquid can drain out. The top of the compartment can be made to have a slanted design to prevent the buildup of moisture within the compartment. Heating elements 140 can provide for heating the air within the compartment to hasten the drying of the mask. The heating elements can be controlled to achieve the desired temperature within the compartment. Lights 145 can illuminate the inside for visibility. The spray and drying cycles can be used to clean the used mask as well as increase the useful life of the mask.

The apparatus may also include suitable irradiation means to sterilize the mask. Examples of such irradiation means include ultraviolet lights 150. A suitable number of ultraviolet lights can be provided within the compartment for irradiating the different surfaces inside the compartment of the apparatus.

Different controls in the form of switches and buttons can be provided on the outer side of the compartment that can be used to control different processes of the apparatus, such as turning on the UV bulbs, spraying antimicrobial solution, setting the drying cycles, setting temperature, defining spray speed and pattern, timer, and the like. Besides the hard controls, the disclosed apparatus can also connect to an external computing device, such as a smartphone. An interface can be provided on the external device that allows a user to interact with the disclosed apparatus, including monitoring the progress of a process and providing different inputs to manipulate one or more settings of the apparatus.

It is also envisioned that the disclosed apparatus can also support features of self-cleaning. The apparatus can self-clean itself. For example, the interiors of the compartments can be washed with the antimicrobial solution and the dry air from the fans and heater can dry up the compartment. The UV lights can also help keep the compartment sterilized and prevent the growth or build-up of microbes.

Above embodiments have been described treating the used masks to extend their useful life, however, treating the new masks is within the scope of the present invention. It is envisioned that the new masks, during production or postproduction, can be treated with antimicrobial solution for providing an antimicrobial coating. Also, any other coating that can affect the utility or life of the mask is also within the scope of the present invention. The antimicrobial coatings, independent of the material or pore size of the masks, can provide enhanced protection against pathogens. The coatings can also be used to change pore size of the mask.

The disclosed apparatus having the nozzles can be controlled by an intelligent unit, wherein the intelligent unit can control the spray droplet sizes, spray volumes, and drying times. Such variations can be used to control the utility of the mask, such as filtration efficiency.

The cleaning and reusing of the mask have several advantages. The adequate stock of the mask can help support humanitarian efforts to countries that don't have adequate supply or access to suggested medical masks by providing a way to extend mask use and providing a way to add a protective antimicrobial for more protection for health care workers and patients. The commercial viability can provide specific mask protective sanitizers, disinfectants, and antimicrobials through an “onsite” solution to the immunocompromised in cancer centers or other specialty acute care.

In certain implementations, the disclosed apparatus can also provide alerts and signals indicating different stages of the process and any malfunction in the system. for example, indicator lights disposed on outside wall of the apparatus can provide alerts and indications. An on or blinking light may indicate that the fluid reservoir is near to empty and needs refill. Similarly, another light may indicate any other service that may be needed. A timer can also be provided that shows time elapsed or left in the completion of the process. Completion and progress of the process can also be shown by colored lights. Besides the light indicators, audio alerts and alarms can also be used.

The disclosed system may also support self-diagnosis features that may help the apparatus to diagnose any fault or potential fault. The disclosed apparatus may also support IoT technologies and the like. Information including alerts can be sent by the disclosed system to external devices through a wired or wireless connection. For example, the apparatus can connect through Wi-Fi, LTE, and the like network technologies. The information may include the number of uses per day, fluid levels, and other operational data.

In certain implementation, the apparatus may also be used with other cleaning, sanitizing, and disinfecting liquids in the processes. The apparatus may use a wide variety of nozzles or systems to create steam, vapors, and different droplet sizes for application of fluids. The apparatus may include a self-cleaning mode or cleaning instructions to be done after a certain amount of uses.

The disclosed apparatus may be provided with an operating system and an interaction panel that provides the user preset spraying and drying cycles for different types of mask materials. The apparatus will also have a digital user ID tracking interface in order to track use of the apparatus.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. An apparatus for extending a useful life of at least a mask, the apparatus comprises:
a compartment having inner walls and a door, the inner walls comprise a bottom wall, a top wall, and side walls;
hooks and/or clips coupled to the inner walls, top wall, and/or bottom wall of the compartment;
a plurality of nozzles configured to spray solution, the plurality of nozzles disposed within the compartment;
one or more fans to blow air mounted within the compartment; and
one or more heating elements to heat air within the compartment.

2. The apparatus according to claim 1, wherein the apparatus further comprises:
one or more ultraviolet lights disposed within the compartment.

3. The apparatus according to claim 1, wherein the compartment has vent holes for air to egress from the compartment.

4. The apparatus according to claim 1, wherein a top portion of the compartment is slanted in design to prevent build-up of moisture.

5. The apparatus according to claim 1, wherein the apparatus further comprises:
a reservoir containing the antimicrobial solution, the reservoir connected to the plurality of nozzles.

6. The apparatus according to claim 1, wherein the door is coupled using hinge joints.

7. A method for extending a useful life of at least a mask, the method comprising:
providing an apparatus comprising:
a compartment having inner walls and a door, the inner walls comprise a bottom wall, a top wall, and side walls,
hooks and/or clips coupled to the inner walls, the top wall, and/or bottom wall of the compartment,
a plurality of nozzles configured to spray solution, the plurality of nozzles disposed within the compartment,
one or more fans to blow air mounted within the compartment, and
one or more heating elements to heat air within the compartment;
mounting a mask to the hooks and/or clips;
spraying the solution over the at least a mask through one or more of the plurality of nozzles; and
upon spraying and after a predefined duration, drying the at least a mask using air from the one or more fans.

8. The method according to claim 7, wherein the apparatus further comprises:
one or more ultraviolet lights disposed within the compartment, the method further comprises:
irradiating the mask with UV light from the one or more ultraviolet lights.

9. The method according to claim 7, wherein the compartment has vent holes for air to egress from the compartment.

10. The method according to claim 7, wherein a top portion of the compartment is slanted in design to prevent build-up of moisture.

11. The method according to claim 7, wherein the apparatus further comprises:
a reservoir containing the antimicrobial solution, the reservoir connected to the plurality of nozzles.

12. The method according to claim 7, wherein the door is coupled using hinge joints.
